(19) United States
(12) Patent Application Publication
(10) Pub. No.: US 2004/0209383 A1
(43) Pub. Date: Oct. 21, 2004

(54) Lift-off process for protein chip

(75) Inventors: Li-Te Yin, Taipei (TW); Jia-Huey Tsao, Taoyuan (TW); Chung-We Pan, Pingtung (TW); Zu-Sho Chow, Hsinchu (TW); Chao-Yun Tsao, Taipei (TW)

Correspondence Address: SUGHRUE MION, PLLC, 2100 PENNSYLVANIA AVENUE, N.W., SUITE 800, WASHINGTON, DC, 20037, US

(73) Assignees: INDUSTRIAL TECHNOLOGY RESEARCH INSTITUTE; CO-WEALTH MEDICAL SCIENCE & BIOTECHNOLOGY, INC.

(21) Appl. No.: 10/417,144

(22) Filed: Apr. 17, 2003

(51) Int. Cl. G01N 33/543

(52) U.S. Cl. 436/518

(57) ABSTRACT

A protein chip and the preparation thereof. The preparation includes providing a substrate with a positive photoresist thereon, forming a spot array valley in the positive photoresist using a spot pattern mask, forming an adhesive layer on the substrate and filling the spot array valley thereon, performing a full region exposure on the positive photoresist and removing the same, thereby leaving the adhesive layer as a spot array pattern on the substrate, and forming an immobilizing material cover the surface of the spot array pattern to obtain a 3-dimensional structure of the immobilizing material.

### BACKGROUND OF THE INVENTION

1. Field of the Invention

The present invention relates to a method for the fabrication of a protein chip. More particularly, the present invention relates to a lift-off process for the fabrication of a protein chip.

2. Description of the Related Arts

Microfabrication techniques, advanced in electronic industry, have been created new opportunities for biochemistry and medical science. Recent progress in microfabrication and micromachining is transforming the field of solid-state transducers into what has become known as microelectromechanical systems (MEMS). MENS technology is currently enjoying a moment of formidable expansion in the health sciences, giving rise to the notion of MEMS for biomedical applications—BioMEMS. As well, as an emerging field in life sciences, proteomics is based upon, and being developed beyond, genomics. Proteins play a central role in establishing the biological phenotype of organisms. Thus proteins are more indicative of functionality whereas the mRNA transcripts are indicative of genetic information, and hence proteomics have become a major focus in life sciences. The protein chip, which uses the proteins to identify specific molecules, is designed for use in handheld devices that will quickly detect low levels of harmful or therapeutic chemicals and microbes. However, protein chips suffer from insufficient detection limits, caused by low signal/noise ratio. To perform better passivation of the array surface is critical in protein chip design.

### SUMMARY OF THE INVENTION

It is therefore a primary object of the present invention to provide a method for the fabrication of a micro-patterned protein chip by lift-off MEMS technology. The method comprises providing a substrate with a positive photoresist thereon, forming a spot array valley in the positive photoresist using a spot pattern mask, forming an adhesive layer on the substrate and filling the spot array valley thereon, performing a full region exposure on the positive photoresist and removing the same, thereby leaving the adhesive layer as a spot array pattern on the substrate, and forming an immobilizing material over the surface of the spot array pattern to obtain a 3-dimensional structure of immobilizing material.

Another object of the present invention is to provide a micro-patterned protein chip developed by lift-off MEMS technology. The protein chip comprises a substrate, a spot array pattern of an adhesive layer thereon, an immobilizing material attached to the spot array pattern, and a biomaterial over the surface of the spot array pattern via the immobilizing material. The immobilizing material displays as a 3-dimensional structure and offers more space for biomaterial to be immobilized. In addition, without the immobilizing material, the biomaterial cannot be immobilized on the substrate, therefore, the area around the spot array pattern of the adhesive layer is devoid of biomaterial. This improves the performance of the signal/noise ratio during the protein chip assay.

### BRIEF DESCRIPTION OF THE DRAWINGS

The present invention will be more fully understood and further advantages will become apparent when reference is made to the following description of the invention and the accompanying drawings in which:

FIG. 1A˜1F are a series of cross sections showing a preferred embodiment of the present invention; FIG. 1D′ is a top view of FIG. 1D.

FIG. 2 is microscopic photograph showing micro-patterning spots.

FIG. 3A˜3B are fluorescence scanning results of SA-Cy5 immobilization on (3A) micro-patterning nitrocellulose chip and (3B) plane nitrocellulose chip.

### DETAILED DESCRIPTION OF THE INVENTION

Without intending to limit in any manner, the present invention will be further illustrated by the following description.

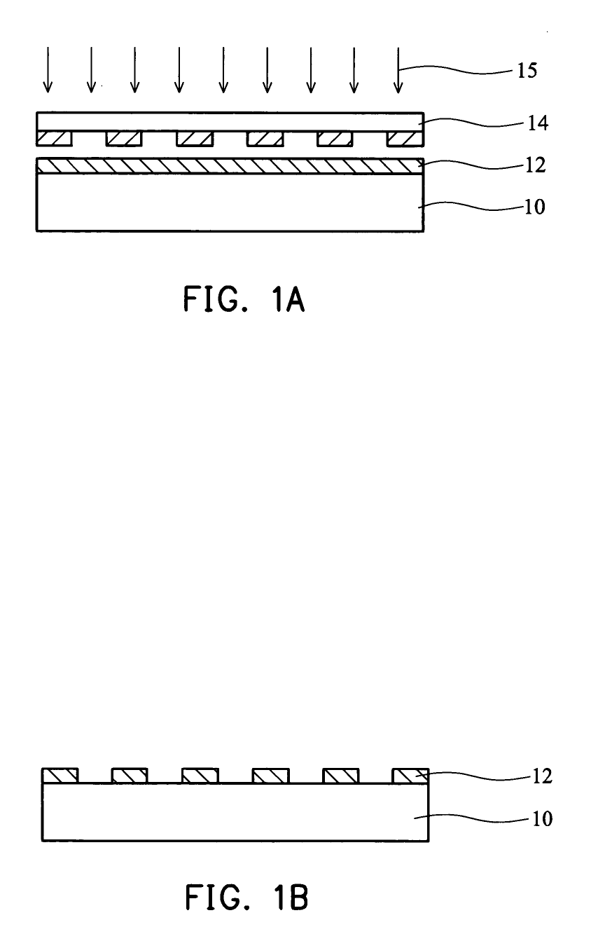

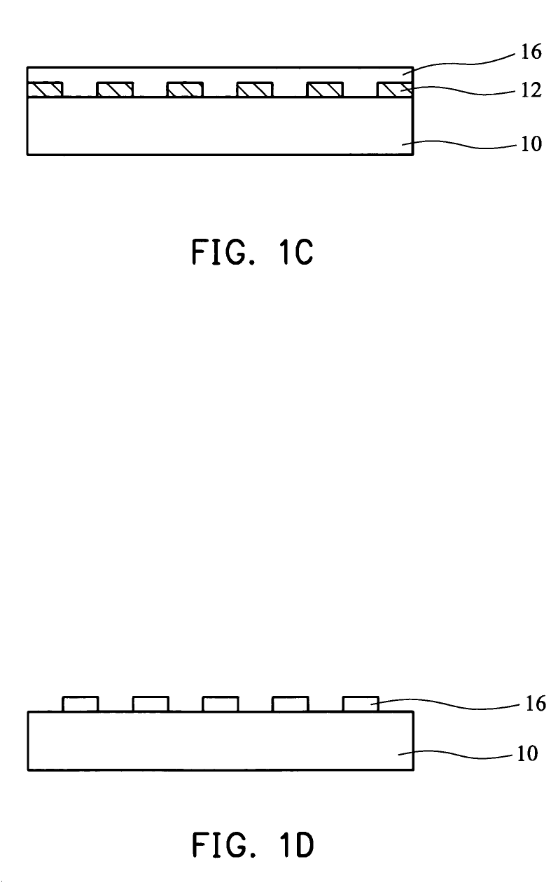

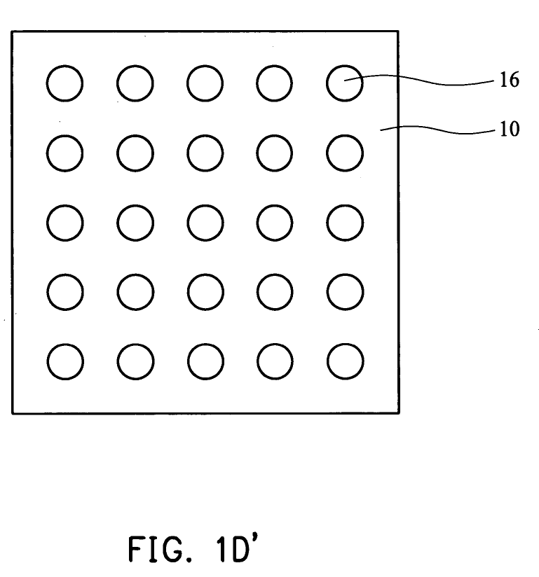

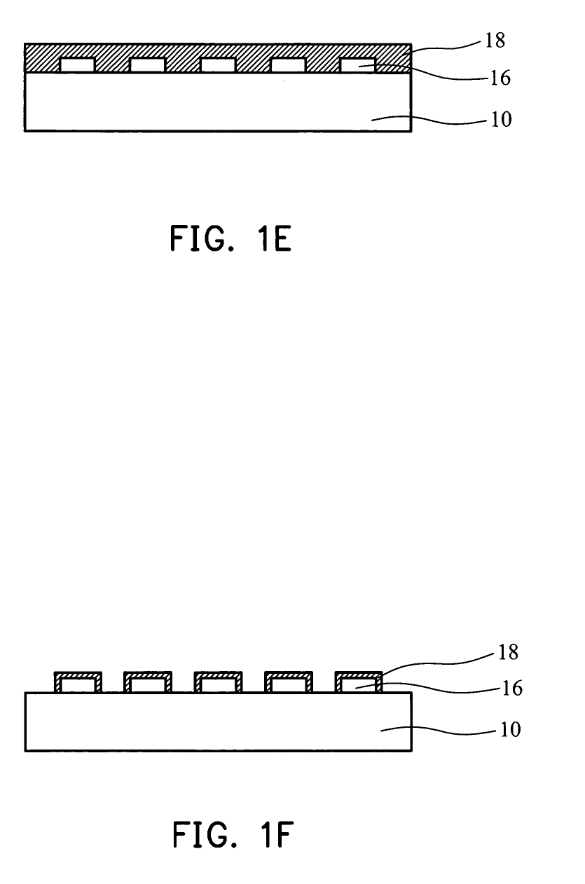

As shown in FIG. 1A, positive photoresist 12 is formed, for example, by spin-coating, on substrate 10. The substrate can be inorganic or organic. Suitable inorganic material includes silicon, silica, quartz, ceramic material, glass, controlled pore glass, carbon, germanium, silicon nitride, zeolites, gallium arsenide, metal such as gold, platinum, aluminum, copper, titanium, and their alloys, or metal oxide such as alumina, titanium dioxide. Suitable organic material includes a polymer polymerized by organic monomers such as ethylene, styrene, propylene, ester, acrylic acid, acrylate, alkyl acrylic acid, or alkyl acrylate. Examples of polymers include, but are not limited to, polystyrene, poly(tetra)fluorethylene, (poly)vinylidenedifluoride, polycarbonate, polymethylmethacrylate, polyvinylethylene, polyethyleneimine, poly(etherether)ketone, polyoxymethylene (POM), polyvinylphenol, polylactides, polymethacrylimide (PMI), polyalkenesulfone (PAS), polyhydroxyethylmethacrylate, polydimethylsiloxane, polyacrylamide, polyimide, co-block-polymers, and Eupergit (R). Photoresists, polymerized Langmuir-Blodgett films, and LIGA structures may also serve as substrates in the present invention.

Next, photoresist 12 is patterned by lithography to develop a spot array valley as shown in FIG. 1B. This can be accomplished by UV exposure 15 using spot array pattern mask 14 and then removing photoresist 12 exposed under spot array pattern mask 14. The thickness of photoresist 12 is 2-5 μM. The spot array pattern of mask 14 can be designed as needed. Each spot of the spot array pattern is circular, square, rectangular, or triangular, preferably circular. The distance between each spot in one row or one line is 50-300 μM.

After that, as shown in FIG. 1C, adhesive layer 16 is formed on substrate 10 and fills the spot array valley of photoresist 12 by spin-coating. The adhesive layer is preferably epoxy resin. The thickness of the adhesive layer is 1-3 μM.

Next, as shown in FIG. 1D and 1D′, A full region exposure is performed, and photoresist 12 and adhesive layer 16 indirectly contacts with substrate 10 are removed. This results in a spot array pattern of adhesive layer 16 on substrate 10 as shown in FIG. 1D′, the top view of FIG. 1D.

Then, as shown in FIGS. 1E and 1F, immobilizing material 18 is formed to cover the surface of spot array pattern of adhesive layer 16 by spin-coating and ultrasonic wash. Immobilizing material 18 is formed on substrate 10 until spot array pattern of adhesive layer 16 is covered as shown in FIG. 1E. The thickness of immobilizing material 18 is 1-5 μM. An ultrasonic wash is then performed to wash out the immobilizing material indirectly contacts with spot array pattern of adhesive layer 16 in order to obtain a structure as shown in FIG. 1F. The spot array pattern of adhesive layer 16 is used as an interlayer for immobilizing material 18. The immobilizing material can be hydrophobic or hydrophilic organic material, preferably hydrophobic organic material such as polystyrene(PS), nitrocellulose(NC), or polyvinylidenedifluoride(PVDF). After ultrasonic wash, any biomaterial of interest can be attached to immobilizing material 18 to obtain a protein chip. The biomaterial includes peptide, protein, antigen, antibody, enzyme, or specific biochemical complex.

With the structure of the present invention, the immobilizing material displays as a 3-dimensional structure and offers more space for biomaterial to be immobilized. In addition, the adhesive layer only appears in a spot array pattern. Without the adhesive layer, the biomaterial cannot be immobilized on the substrate via the immobilizing material. Therefore, the biomaterial also shows as a spot array pattern, and the area around the spot array pattern of the adhesive layer is devoid of the biomaterial. The contamination of the background is, thus, effectively reduced. This improves the performance of the signal/noise (S/N) ratio during the protein chip assay.

### EXAMPLE

Material and Methods

Preparation of Micro-Patterned Arrays:

The micro-patterned protein chip was developed by applying lift-off MEMS technology, and formed based on a glass substrate and a positive photoresist to develop a spot array valley by UV exposure with a spot array pattern mask. Epoxy resin was then formed on the substrate, filling the spot array valley by spin-coating. A full region exposure and development for glass substrate was executed to remove the epoxy resin indirectly contacts with glass substrate. Thus, a spot array pattern of the epoxy resin was developed, used as interlayer for immobilizing material (nitrocellulose). Finally, a micro-patterning of immobilizing polymer was formed by spin-coating and ultrasonic wash.

Protein Immobilization

Fluorescence conjugated protein, streptavidin-Cy5 (20 μg/mL), was used to examine the immobilization rate between polymer spot and glass substrate.

Results

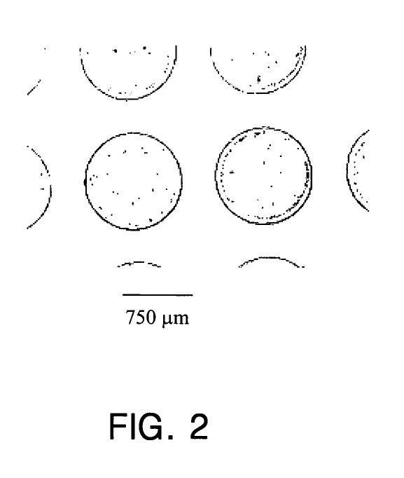

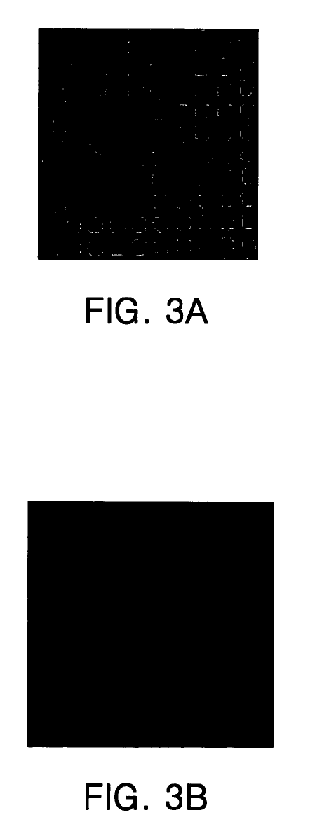

FIG. 2 shows microscopic photograph of the micro-patterning spots. The circle structure of nitrocellulose spot pattern is still intact after lift-off fabrication. The circle in the lithographic mask was designed to a diameter of 700 μm; however, the micro-patterning spots show a diameter of 750 μm after the lift-off fabrication. Nitrocellulose covered on the adhesive layer causes larger patterns of the spots. The results of the protein immobilization examined by immobilizing SA-Cy5 on micro-patterning nitrocellulose chip and plane nitrocellulose chip are shown as FIGS. 3A and 3B, respectively. The spot signals are clear in the micro-patterned chip (FIG. 3A). The results indicate that the micro-patterned protein chip supports better S/N ratio than traditional plane chip and this is valuable in low signal protein affinity assay.

While the invention has been particularly shown and described with the reference to the preferred embodiments thereof, it will be understood by those skilled in the art that various changes in form and details may be made without departing from the spirit and scope of the invention.

1. A method of fabricating a protein chip, comprising the steps of:
(a) providing a substrate with a positive photoresist thereon;
(b) forming a spot array valley in the positive photoresist using a spot pattern mask;
(c) forming an adhesive layer on the substrate and filling the spot array valley thereon;
(d) performing a full region exposure on the positive photoresist and removing the positive photoresist, thereby leaving the adhesive layer as a spot array pattern on the substrate;
(e) forming an immobilizing material over the surface of the spot array pattern to obtain a 3-dimensional structure of the immobilizing material; and
(f) immobilizing a biomaterial on the substrate via the immobilizing material.

2. (canceled).

3. The method as claimed in claim 1, wherein the substrate comprises an inorganic material of silicon, silica, quartz, ceramic material, glass, controlled pore glass, carbon, germanium, silicon nitride, zeolites, gallium arsenide, metal or metal oxide.

4. The method as claimed in claim 1, wherein the substrate comprises a polymer resulting from the polymerization of organic monomers, wherein the organic monomers are ethylene, styrene, propylene, ester, acrylic acid, acrylate, alkyl acrylic acid, or alkyl acrylate.

5. The method as claimed in claim 1, wherein the positive photoresist is spin-coated on the substrate.

6. The method as claimed in claim 1, wherein the adhesive layer is epoxy resin.

7. The method as claimed in claim 1, wherein the adhesive layer of step (c) is formed by spin-coating.

8. (canceled).

9. The method as claimed in claim 1, wherein the immobilizing material is hydrophobic.

10. The method as claimed in claim 9, wherein the immobilizing material is polystyrene (PS), nitrocellulose (NC), or polyvinylidenedifluoride (PVDF).

11. The method as claimed in claim 1, wherein step (e) is performed by spin-coating and ultrasonic wash.

12. The method as claimed in claim 12, wherein the biomaterial comprises antibody, antigen, polypeptide or enzyme.

13. A protein chip fabricated by lift-off technology, comprising:
a substrate;
a spot array pattern of an adhesive layer thereon;
an immobilizing material attached to the spot array pattern; and
a biomaterial covering the surface of the spot array pattern via the immobilizing material.

14. The protein chip as claimed in claim 13, wherein the substrate comprises an inorganic material of silicon wafer, ceramic material, glass, or metal.

15. The protein chip as claimed in claim 13, wherein the substrate comprises a polymer polymerized by organic monomers, wherein the organic monomers are ethylene, styrene, propylene, ester, acrylic acid, acrylate, alkyl acrylic acid, or alkyl acrylate.

16. The protein chip as claimed in claim 13, wherein the adhesive layer is epoxy resin.

17. The protein chip as claimed in claim 13, wherein the immobilizing material is hydrophilic.

18. The protein chip as claimed in claim 13, wherein the immobilizing material is hydrophobic.

19. The protein chip as claimed in claim 18, wherein the immobilizing material is polystyrene (PS), nitrocellulose (NC), or polyvinylidenedifluoride (PVDF).

20. The protein chip as claimed in claim 13, wherein the biomaterial comprises peptide, protein, antibody, antigen, enzyme, or specific biochemical complex.
